(19) United States
(12) Patent Application Publication
(10) Pub. No.: US 2003/0002685 A1
(43) Pub. Date: Jan. 2, 2003

(54) Electronic stethoscope

(76) Inventor: Marc S. Werblud, Seattle, WA (US)

Correspondence Address: CHRISTOPHER A. WIKLOF, 3531 99TH STREET S.E., EVERETT, WA, 98208, US

(21) Appl. No.: 09/894,616

(22) Filed: Jun. 27, 2001

(51) Int. Cl. A61B 7/04; A61B 7/02; A61B 5/02; A61B 7/00

(52) U.S. Cl. 381/67; 181/131; 600/528; 600/586

(57) ABSTRACT

An electronic auscultation aid improves the performance and convenience of auscultation. The auscultation aid may include means for emphasizing sounds that may be associated with pathological or pre-pathological conditions. The auscultation aid may include a wireless interface for distributing a detected signal to one or more remote devices. The auscultation aid may comprise a mount for temporary attachment to a conventional acoustic stethoscope. The auscultation aid may include an apparatus for switching between active and sleep modes for the conservation of battery life.

### FIELD OF THE INVENTION

The present invention relates to stethoscopes and other auscultation devices, and more particularly to the field of electronic stethoscopes.

### BACKGROUND OF THE INVENTION

Historically, a physician might have placed his ear against a patient's chest in an effort to detect suspicious sounds. The practice of listening to sounds emanating from a patient and drawing diagnoses or making a health assessment therefrom is called auscultation, the verb form being to auscultate. Unaided auscultation was an effective enough practice that the acoustic stethoscope was invented.

A conventional acoustic stethoscope conveys sound from inside a patient to the ears of a physician or other health-care provider via acoustic tubes that isolate the signal. The acoustic tubes are coupled to the patient's body through a bell that may optionally be covered by an acoustic diaphragm. Stethoscopes are useful in that they both spatially isolate sounds and, through the use of an enlarged bell or parabola-shaped sound collecting area, may help to amplify sounds to a level high enough to distinguish over the ambient noise. Frequently, modern acoustic stethoscopes have two bells, each having a different diameter. The larger, diaphragm-covered bell is often used for general practice while the smaller diameter bell, frequently not covered by a diaphragm, is often used to distinguish more subtle sounds such as heart sounds.

In the medical and health care arts, auscultation is combined with other practices to determine the health of a patient. In the case of a general or family practice, the stethoscope user must train for many hours to identify a plethora of possible pathological or pre-pathological conditions. Frequently, the identification by auscultation of obscure conditions or conditions that are accompanied by relatively obscure audio signs or signals is beyond the competence of general practice physicians and nursing personnel. In these cases, specialists may be able to readily identify the sound of a pathology that is in their field. Unfortunately, it is not practical, for a patient not having a known pathology, to consult specialists to auscultate for every possible condition in the hope of early discovery of any possible disease condition. Because of the need to control health care costs, it is often necessary for a disease process to mature sufficiently to exhibit itself in an overt manner prior to consulting with a specialist who otherwise may have been able to detect the incipient condition earlier through practiced auscultation, and/or detailed testing, for instance by echocardiogram.

In the field of stethoscope design, there has recently been an emergence of a variety of electronic stethoscopes. Examples are described in issued U.S. Pat. Nos. 5,844,995; 5,852,263; 5,932,849; 6,002,777; 6,005,951; 6,026,170 and 6,028,942, all hereby incorporated by reference. The electronic stethoscopes disclosed in the prior art share a common ability to amplify a detected signal. Additionally, some of these stethoscopes are equipped with audio equalizers to increase the relative volume of a pre-determined portion of the audio spectrum.

Many current electronic stethoscopes are susceptible to failure in the event of electronic or battery failure. This can pose a significant inconvenience to the user or even render the instrument useless.

### OVERVIEW OF THE INVENTION

In one embodiment of the present disclosure, applicant seeks to provide an apparatus for converting a conventional acoustic stethoscope to an electronic stethoscope, that apparatus being especially well adapted for providing the benefits of electronic signal processing to auscultation.

In one aspect, an auscultation aid may be removably attached to a wide variety of acoustic stethoscopes by use of a mounting system that is adaptable to a variety of device form factors.

In another aspect, an electronic stethoscope may comprise an acoustic stethoscope with an electronics portion that may be inserted into an air column between the bell and earpiece.

In another aspect, the electronic stethoscope may have equalization circuitry. The equalization circuitry may help detect pathological or pre-pathological conditions.

In another aspect, the electronic stethoscope may have provision for transmitting a signal to other devices.

In another aspect, the electronic stethoscope may transmit a signal representing a detected audio signal to a computer, the computer may process the received signal and may transmit a signal back to the electronic stethoscope, whereby results of the processing are made apparent to the user.

In another aspect, the detected audio signal may be processed so to increase the relative acoustic amplitude of a portion of the audio signal in a manner intended to draw the users' attention to possible pathological or pre-pathological conditions.

In another aspect, the relative acoustic amplitude of a portion of the audio signal may be altered as a function of comparison to a library of standard audio signals. The library of standard audio signals may comprise signals characteristic to a plurality of pathological and pre-pathological conditions.

In another aspect, an electronic stethoscope has a profile library stored internally for comparing a newly acquired signal thereto, the profile library comprising a plurality of standard audio signals.

In another aspect, an electronic stethoscope may have provision for sharing output between a plurality of listeners.

In another aspect, an electronic stethoscope adapter may have provision for recording and playback.

In another aspect, an electronic stethoscope may have controls for facilitating easy and intuitive operation by a user and for selecting manual or automatic operation of electronic features.

In another aspect, recommended or optimal instrument placement may be indicated to the user.

In another aspect, a system includes a means for comparing a stored periodic audio signal to a newly acquired audio signal, substantially irrespective of non-linked characteristics such as body size, heart rate, respiration rate, age, sex, and other individual idiosyncrasies of the individual from whom the new audio signal is detected.

In another aspect, known patient characteristics may be used in combination with a detected audio signal to compare to a variety of standard pathological signals.

In another aspect, an electronic stethoscope has a means for manually sampling a variety of standard pathological signals for a user to compare against a newly acquired signal.

In another aspect, an electronic stethoscope includes a manual trigger for storing a newly acquired audio signal, the trigger causing the storage of the signal from a time preceding the activation of the trigger.

In another aspect, an electronic stethoscope includes an automatic trigger for storing a newly acquired audio signal, the automatic trigger being enabled by positive comparison of the new signal to one of a plurality of stored signals in an audio signal library.

In another aspect, an electronic stethoscope may comprise an electronics portion for detecting an audio signal and converting it to an electrical signal, an interface for carrying the electrical signal, and a headphone or earpiece that receives the electrical signal from the interface and converts it into an audio signal.

### BRIEF DESCRIPTION OF THE DRAWINGS

FIG. 1A is a cross-sectional depiction of one embodiment where a compressible toroidic member temporarily affixes an electronics housing to a bell of a conventional acoustic stethoscope.

FIG. 1B is a partial cross-section corresponding to FIG. 1A showing the adaptability of the compressible toroid to an acoustic stethoscope having a shape different than that of FIG. 1A.

FIG. 1C shows cross-sections of optional interchangeable compressible toroidic members for adaptation to a range of bell shapes. One of the interchangeable compressible toroids illustrates an optional pressure-sensitive adhesive feature for semi-permanently mounting into a receiving bore of the electronics housing.

FIG. 2 is a perspective view of a compressible toroidic member having pressure-sensitive adhesive thereon.

FIG. 3 is a cross-sectional view of an alternative adaptable receiving bore for a stethoscope bell where the bell is retained by levers spring-mounted on the receiving bore.

FIG. 4 is a cross-sectional view of an alternative adaptable receiving mechanism for a stethoscope bell using a strap having variable effective diameter.

FIG. 5 is a perspective view of a receiving means that uses a strap to attach an electronics enclosure to a stethoscope bell.

FIG. 6 is a cross-sectional view of an alternative adaptable receiving mechanism for a stethoscope bell that uses set-screws to retain the bell.

FIG. 7 is a cross-sectional view of an alternative mounting mechanism that uses an insertion member mounted on a stethoscope bell for mating with a corresponding receiving member on an electronics housing.

FIG. 8 is a cross-sectional view of an alternative mounting mechanism that uses a bracket that may be permanently or semi-permanently mounted on a stethoscope.

FIG. 9 is a cross-sectional view of an alternative mounting mechanism that uses magnets, attached to the housing of the auscultation aid, for magnetically attaching to the bell of a stethoscope. FIG. 9 also shows magnets or ferromagnetic components semi-permanently attached to the stethoscope bell for coupling with the housing-mounted magnets.

FIG. 10 is a simple block diagram of some basic components of one embodiment of an electronic stethoscope.

FIG. 11 is a block diagram of a more sophisticated electronic stethoscope having signal processing capabilities.

FIG. 12 is a block diagram of a detection device for automatically switching between power saving and full power states depending upon proximity to an auscultation subject.

FIG. 13 is a diagram of power profiles corresponding to use and stand-by conditions.

FIG. 14 is a flow chart illustrating a method for automatically switching the device between “on” and “power-saving” modes.

FIG. 15 is a block diagram of an embodiment having connectivity to remote devices.

FIG. 16 is a flow chart illustrating a method for comparing an auscultation sample signal to a library of signals characteristic of pathological and pre-pathological conditions, and presenting sample matches to a user.

FIG. 17 is a flow chart illustrating a method for enhancing the aspects of a sample auscultation signal that are non-nominal (termed a residual signal), and therefore may be associated with a pathological or pre-pathological condition.

FIG. 18 is a block diagram of an electronics embodiment.

FIG. 19 illustrates one embodiment of a user interface of an auscultation aid.

FIG. 20 is a side view of the embodiment of FIG. 19.

FIG. 21 is an isometric view of the embodiment of FIGS. 19 and 20.

FIG. 22 is a diagram of an electronic auscultation aid that may be inserted into the air column of an acoustic stethoscope.

FIG. 23 is a perspective view of an electronic auscultation system comprising a detector portion and a headphone portion.

FIG. 24 is a block diagram of an electronic auscultation system having a detector portion and an electronically coupled earpiece portion.

### DETAILED DESCRIPTION OF THE INVENTION

As indicated above, certain attributes of the present invention relate to an electronic stethoscope or an electronic auscultation enhancement module that may be used to convert a conventional acoustic stethoscope to electronic operation. In some embodiments, some or all of the electronics module may be removably mounted to the bell of an acoustic stethoscope.

Turning now to the accompanying figures, we will examine some particular aspects of various embodiments of the present invention.

A numbering convention to facilitate easy understanding by the reader is used herein. Figures are numbered in conventional consecutive order. Specific features are generally indexed consecutively using three or four digit numbers in the order described. The first one or two digits correspond to the figure number in which the feature is first described. Features having similar functionality generally retain their originally assigned number throughout, even though their physical or logical appearance may vary considerably from figure to figure.

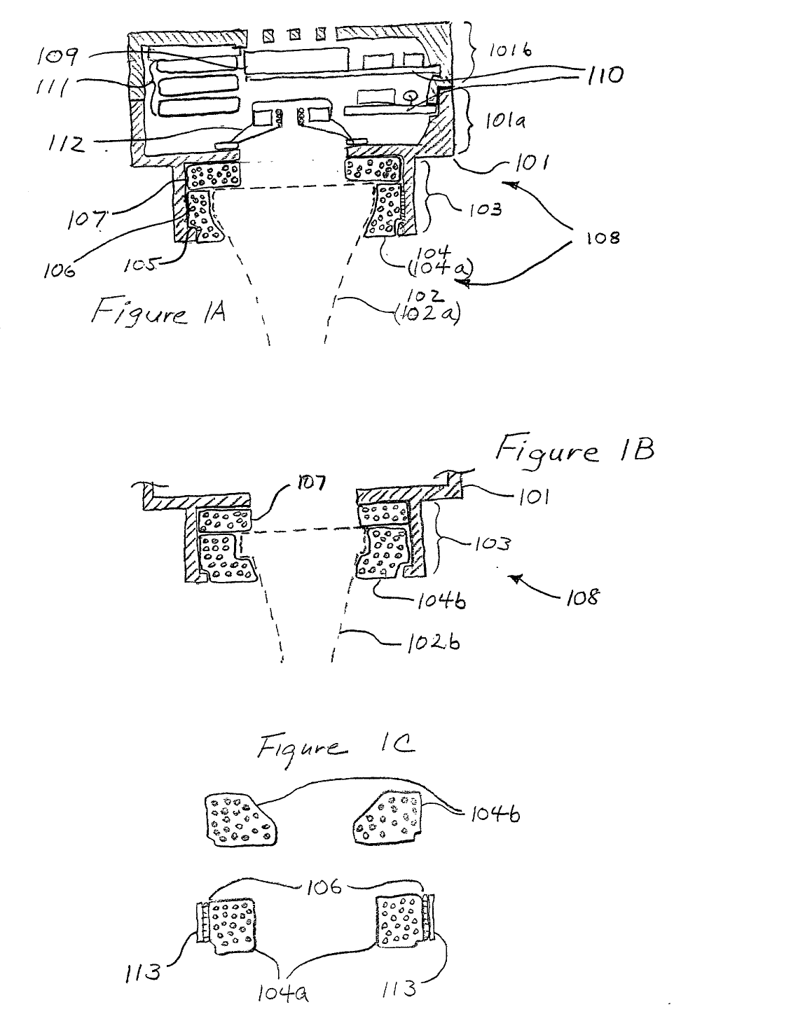

FIG. 1A, a cross-sectional view of one embodiment, shows several functional elements including a mechanism for removably mounting the electronics module 101 on the bell of a conventional acoustic stethoscope 102, the specific instance of which is denoted as 102a. The electronics module 101 is shown comprising housing halves 101a and 101b enclosing electronic components. A bell mount 108 includes receiving bore wall103, compressible member 104, and sealing gasket 107. A receiving bore wall 103 extends downward from lower housing half 101a and is shaped to receive stethoscope bell 102. A toroidic compressible member 104 contacts and fits the inserted stethoscope bell 102 inside the receiving bore wall 103. The specific instance of compressible member 104 is denoted 104a. Compressible member 104 may be held inside receiving bore wall 103 by compression fit, may optionally gain additional retention by means of retention lip 105 or may optionally be held in place by adhesive layer 106. Sealing gasket 107 may optionally be used to provide enhanced acoustic isolation and provide more positive seating of stethoscope bell 102.

Toroidic compressible member 104 and gasket 107 may be comprised of a variety of materials such as polystyrene foam, natural or synthetic rubber, polybutadiene, closed-cell polyurethane foam, or other compressible material. In certain cases, it may be preferable to form gasket 107 with a higher modulus of elasticity than toroidic compression member 104 to facilitate positive tactile response during insertion of stethoscope bell 102.

To use bell mount 108 with a particular stethoscope having bell 102a, the user axially inserts the bell 102a into the passage of receiving bore 103 and toroidic compression member 104 until seated against gasket 107. This may require some amount of force to overcome the elastic strength of toroid 104 and friction between the surface of stethoscope bell 102 and toroid 104. To overcome the latter, the user may be well served to exert a slight twisting motion while axially inserting the bell. To remove the auscultation aid from the acoustic stethoscope, the user reverses the insertion motion; pulling the bell mount 108 and stethoscope bell apart axially while optionally applying a slight twisting motion.

FIG. 1A also depicts cross-sections of other physical components of the electronic auscultation aid including microphone or detector 109, electronics 110, battery pack 111, and speaker or emitter 112. The interactivity of these portions of the unit will be described in greater detail later. For now, the reader's attention is drawn in part to a preferred position of microphone 109 approximately axial to and opposite stethoscope bell 102, behind a protective grill formed in the upper housing 106b. Speaker 112 is also situated axially with respect to the bell centerline but in the bottom housing 101a and facing bell 102. The relative positions of these components should be governed by ergonomic, environmental, size, cost, weight, physical robustness, and sound fidelity considerations. Not shown in FIG. 1A are user controls or indicators, nor optional input/output (I/O) ports for external signal processing and/or distribution.

FIG. 1B is a cross-sectional view of bell mount 108 adapted to mount a bell 102b having substantially different geometry than bell 102a. In FIG. 1B, elastic toroid 104b has been substituted for elastic toroid 104a, the difference being in the inside diameters of the toroids. Toroid 104b has a smaller and more variable inside diameter than 104a for the purpose of receiving and retaining the substantially smaller outer diameter of stethoscope bell 102b. The elastic nature of receiving toroids of the present embodiment may allow them to accept and retain stethoscope bells having moderate differences in diameter and geometry without the necessity of changing toroids to fit. Indeed, features such as radially or rotationally variable elastic modulus, and/or the inclusion of radial ridges in the foam surface may tend to increase the range of bell diameters easily accommodated by a particular receiving toroid. However, the use of interchangeable receiving toroids may serve to increase the range of compatible stethoscope bell geometries and may help the user to obtain an optimum mechanical coupling.

Receiving toroid 104a further includes optional adhesive for improving the retention of the elastic member in receiving bore 103. Adhesive layer 106, which may be a pressure-sensitive adhesive, is coated in one or more places around the outer diameter of toroid 104a. Release liner 113 may be optionally used to protect adhesive layer 106 during shipping and prior to installation of toroid 104a into receiving bore 103. For cases where a pressure sensitive adhesive is used, it may be preferable for such adhesive to be of a type having moderately low initial tack but relatively high ultimate peel strength relative to the material of receiving bore 103. This combination may help to facilitate easy installation and repositioning for a period of time while maintaining good mechanical robustness during subsequent usage. Pressure sensitive adhesives having suitable properties may be formulated from a natural or synthetic rubber base, acrylic emulsion base, acrylic solution base, block copolymer base, silicone base, or other suitable formulation known to the art. Release liner 113 may be formed from Kraft paper having a silicone release layer, polypropylene, or other material known to the art. In one embodiment, pressure sensitive adhesive 106 and release layer 113 may be formed as transfer adhesive tape where a double-sided release liner and adhesive are self-wound. To apply to the retention toroid, a low release strength side of the release liner releases during unwinding of the tape, the tape is cut to a length corresponding to the outer diameter of the toroid, and the adhesive side is applied to the outer diameter of the toroid.

FIG. 2 is an isometric view of toroid 104a with adhesive 106 and release liner 113 applied thereto. Optional symbology or directions 201 may be printed on the outer surface of release liner 113 to aid the user in installing the toroid.

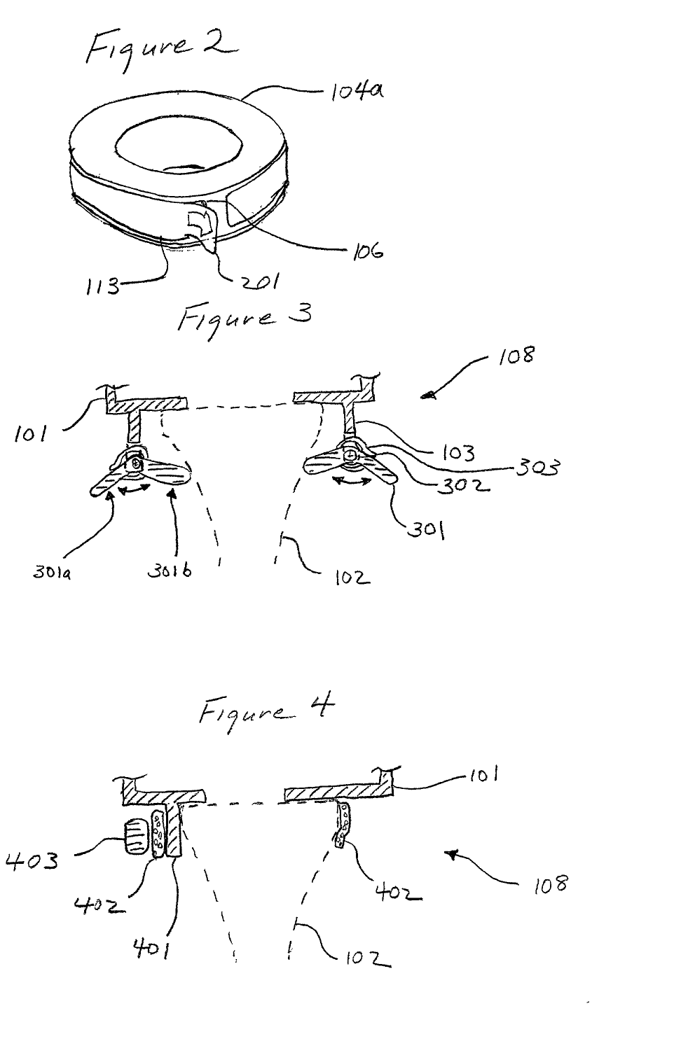

FIG. 3 is a cross-sectional view of an alternative stethoscope bell receiving assembly 108. Boss 103, which may be formed integrally with housing 101, extends therefrom. Optionally, boss 103 may be formed as a continuous receiving bore wall as shown in antecedent figures, or may be formed as one or more non-continuous protuberances from housing 101. Lever 301 is rotatably coupled to boss 103 on shaft 302, and urged to rotate medially by spring 303.

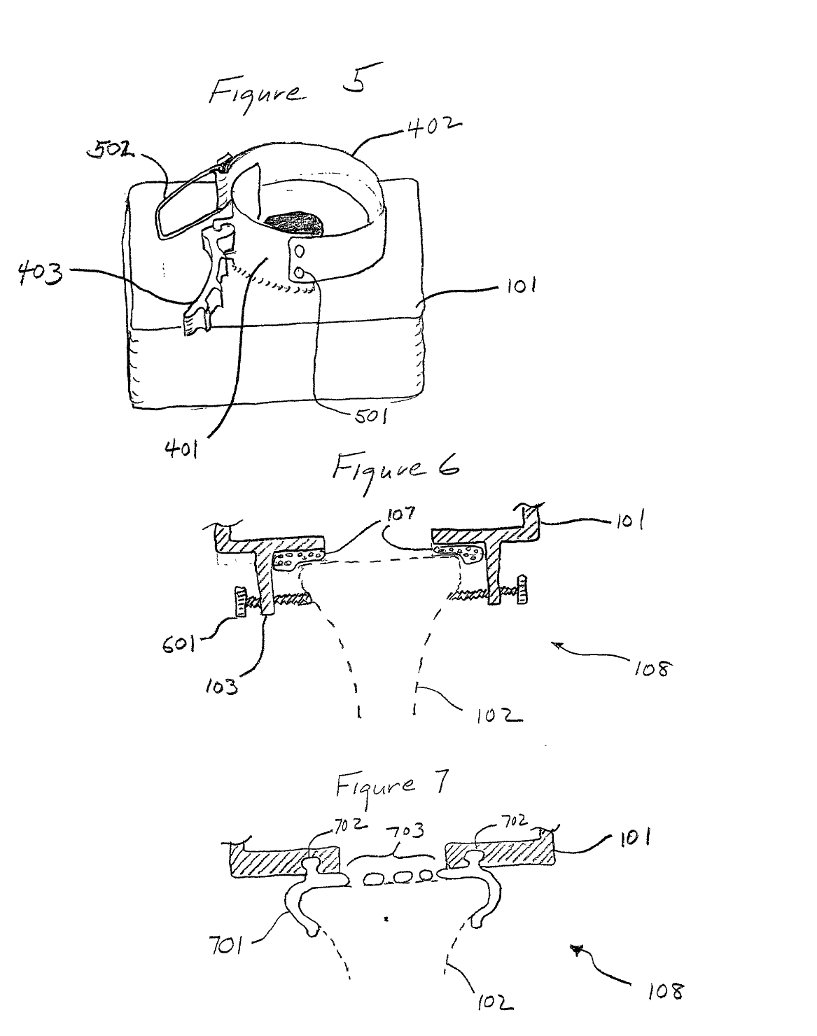

To install auscultation aid on stethoscope bell 102, the user presses the actuation end of lever 301, denoted feature 301a, laterally, thus rotating engagement end of lever 301, denoted feature 301b, up and out of the path of the bell. After inserting bell 102 to a point approximately corresponding to or closer than shaft 302, the user releases actuation end 301a, thereby allowing engagement end 301b to rotate against the underside of bell 102. This urges the face of bell 102 against the surface of housing 101 and temporarily mounts the auscultation aid to the stethoscope. Optionally an acoustic gasket 107 may be formed on the surface of housing 101 to aid in isolation of the air column inside the stethoscope. In the embodiment shown, two lever assemblies are shown for holding the stethoscope bell against the coupling surface of the auscultation aid. In practice, one lever, three levers, or more may be used effectively. Especially if two or fewer lever assemblies are used, it may be advantageous to bifurcate the engagement end of the lever 301b to help keep the stethoscope bell centered or tangent relative to boss 103. If only one lever is used, an indexing feature such as that shown in FIGS. 4 and 5 below can be placed opposite boss 103 to locate the stethoscope bell relative to housing 101 and provide positive engagement for lever 301 against the lower surface of bell 102.

FIG. 4 is a cross-sectional view of another alternative mounting assembly. Indexing feature 401 is formed to one side of the emitter aperture in housing 101. A first end of strap 402 is affixed to the medial surface of the indexing feature and wrapped circumferentially around stethoscope bell 102 such that, when tightened, the bell is held against both the indexing surface and against the coupling surface of housing 101. A buckle 403 may be used to secure the second end of strap 402.

FIG. 5 is an isometric view of the bell mount shown in FIG. 4 showing the first end of strap 402 affixed to indexing feature 401 by rivets 501. Alternatively, strap 402 by be affixed to indexing feature 401 by any number of known means including gluing, integral formation, clamping, heat staking, interference fitting, or any other means known to the art. The second end of strap 402 has a bale 502 fitted thereto for engaging buckle 403.

In some embodiments, strap 402 may be formed from an elastic material such as natural or synthetic rubber, polyurethane, silicone, leather, or other material known to the art. This is useful in that it allows strap 402 to conform to the lower surface of mounted bell 102 (as shown more clearly on the right-hand cross-section of the strap in FIG. 4) which, in turn, helps to urge the bell against the mating surface of housing 101 or optional gasket 107 (not shown). If strap 402 is from a flexible but inelastic material, then elasticity may be gained by means of a bale 502 having integral springs.

FIG. 6 shows an alternative embodiment of bell mount 108 using set screws 601 passing through bosses 103. The set-screws may be conveniently adjusted to press against the underside of bell 102, thus urging it against the mating surface of housing 101 or optional acoustic gasket 107, as shown. As in other embodiments, bosses 103 may be formed as portions of a continuous cylindrical wall or, alternatively, as individual protuberances from housing 101. In a preferred embodiment, setscrews 601 are formed from a relatively soft material such nylon, Noryl, brass, or other material to avoid their tips from scratching the underside of bell 102. Alternatively, soft tips may be installed on setscrews formed from harder materials. In principle, any number of setscrews may be used but a preferred number is three. If only one or two setscrews are used, then an indexing feature 401 should be used opposed the set-screw(s) to create a positive location limit for the stethoscope bell 102.

FIG. 7 is a cross-sectional view of an alternative embodiment for bell mount 108. In this embodiment a pliable cup 701 is used to receive stethoscope bell 102. The pliable elastic cup may be formed, for instance, by injection molding silicone, polyurethane, latex, or other suitable material. This one-piece cup may be affixed to housing 101 via an interference-fit circumferential channel 702 such as shown, or alternatively may use other suitable known means such as gluing, riveting, heat-staking, etc. Pliable cup 701 may additionally be formed monolithically or may be formed from two or more substances by, for instance, co-molding. Mounting cup 701 may include a perforated region 703 to bridge the emitter aperture. While many flexible materials are inherently unstable, this region can help stabilize the cup by providing elasticity across the bell diameter, thus reducing stress load on circumferential channel 702. Insertion and removal of the stethoscope bell may be aided by applying silicone, talc, Teflon powder, or other lubricant to the inside of the cup. Alternatively, the surface of the material may be impregnated with one or more of such materials.

The bell mount 108 of FIG. 7 is typically mounted on the stethoscope by applying a twisting motion between the bell 102 and housing 101 while applying axial force to seat the stethoscope bell. In some instances, it may be useful to fully seat one edge of the bell in the cup and twist the opposite edge in. Removal of the auscultation aid from the stethoscope may be similarly accomplished by applying a twisting motion while applying axial tension between the cup and the stethoscope bell.

FIG. 8 is a cross-sectional view of another alternative embodiment that makes use of a semi-permanent bracket attached to the bell of an acoustic stethoscope, the bracket in turn having provision for mating to features in the housing 101 of the auscultation aid. Bracket 801 is semi-permanently attached to the user's stethoscope bell, optionally using gasket 802 therebetween to obtain a mechanically and acoustically tight fit. Such attachment may be made by a number of known approaches including the use of one or more screws between halves of the bracket, by gluing, or by using heat softening or positive coefficient of thermal expansion characteristics of the material to seat while hot. Once installed on the stethoscope bell, bracket 801 has a known geometry and may be removably attached to housing 101 using a number of known processes.

In the example of FIG. 8, the outer surface of bracket 802 has a threaded coupling and the inner surface of receiving bore 103 has corresponding threads for easy screw-on attachment of the auscultation device. Also evident is optional gasket 107 which may help form a tight acoustic seal and provide a known coefficient of friction against the corresponding surface of bracket 801, gasket 802, and/or the face of stethoscope bell 102. Alternatively, mating surfaces of bracket 801 and receiving bore 103 may be reversed such that bracket 801 is affixed to the inside of bell 102, threads of bracket 801 are on its inside diameter, and mating threads of receiving bore 103 are on its outside diameter. In still another approach, bracket 801 and receiving bore 103 may be formed to create a bayonet-mount or other coupling in place of the screw coupling shown in FIG. 8.

As another alternative to the approach described above with reference to FIG. 8, bracket 801 may be formed integrally with stethoscope bell 102 as a single circumferential feature or as discrete features formed at points around the circumference of stethoscope bell 102. In this instance, such features may be formed for instance by casting, machining, molding, or other means.

Alternatively, the fit between housing 101 and stethoscope bell 102 may be magnetic in nature as shown in FIG. 9. In FIG. 9, a cross-sectional depiction, housing 101 contains one or more magnets, shown as 901a and 901b. For the case of a stethoscope bell comprised of a ferrous material such as chrome-plated steel, these magnets alone may be sufficient to securely but temporarily attach the housing 101 to bell 102. For the case of a non-ferrous stethoscope bell such as one comprised of aluminum or stainless steel, auxiliary coupler 902 may be attached to the bell to provide a material responsive to the magnetic field of magnets 901. In one case, auxiliary coupler 902 may be comprised of a one-piece magnetic strip attached to the bell by means of pressure sensitive adhesive. In another case auxiliary coupler 902 may be comprised of discrete magnets or even of a ferrous material having no magnetic field of its own. Alternatively, auxiliary coupler 902 may be formed integrally with stethoscope bell 102.

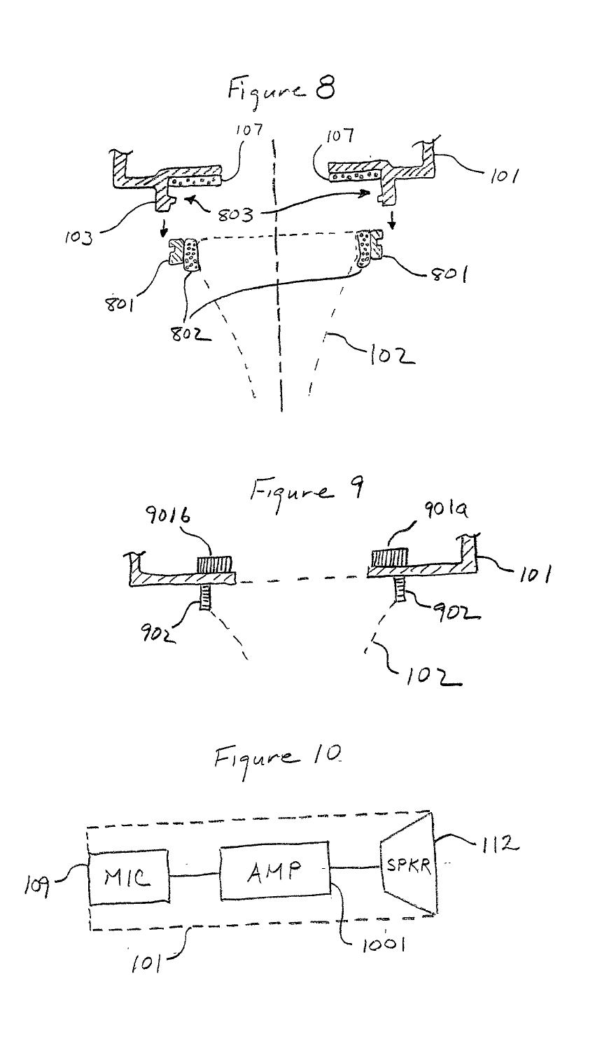

Turning now to the electronics, the block diagram of FIG. 10 shows the main components of one embodiment. Housing 101 contains a microphone 109 carried by housing 101 and positioned to detect sounds for auscultation. Amplifier 1001 amplifies signals from microphone 109. Amplified signals may then be output by speaker 112, positioned so as to emit sounds into an acoustic stethoscope to which housing 101 may be coupled. Speaker 112 may be disposed so as to emit amplified audio power into one or more air columns of a conventional acoustic stethoscope to which auscultation aid 101 may be attached.

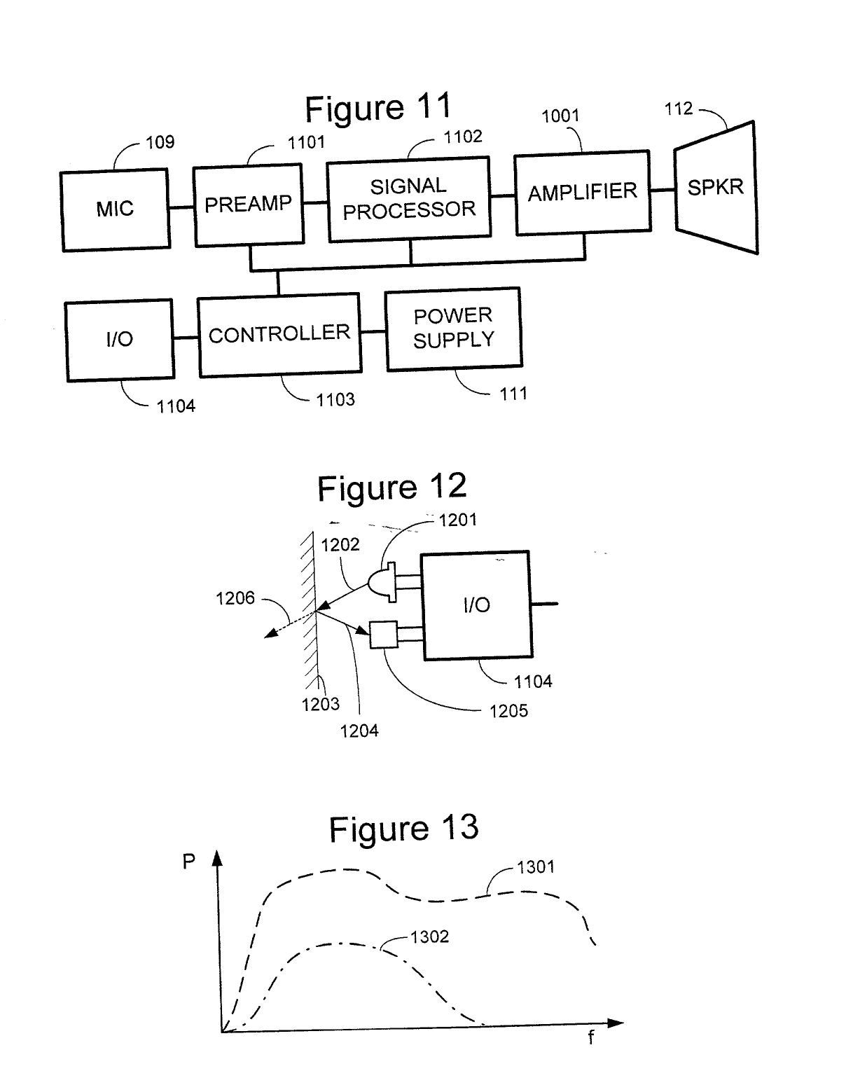

In some embodiments, additional features may be added as shown in FIG. 11. Microphone 109 may be positioned to detect sounds from an auscultation subject. Signals from microphone 109 may be amplified to a usable level by pre-amplifier 1101 which may be a simple pre-amplifier known to the art and may also contain additional functionality such as non-linear amplification, output level limit circuitry, input level detection circuitry, etc. Output from pre-amplifier 1101 may be passed to optional signal processor 1102 which may, for instance, provide functionality such as bandpass filtering, non-linear filtering, output level limiting, power saving circuitry, audio compression, signal augmentation, etc. In addition, signal processor 1102 may include analog-to-digital (A/D) conversion. In certain embodiments, a digital representation of a signal detected by microphone 109 may be passed to a micro-controller 1103. Output from optional signal processor 1102 may be passed to main amplifier 1001, which in turn powers output speaker 112. Signal processor 1102 may comprise, for instance, a digital switch for routing signals between pre-amplifier 1101, controller 1103, and amplifier 1001.

Controller 1103 may control the operation of pre-amplifier 1101, optional signal processor 1102, and amplifier 1001. Input-output (I/O) block 1104 may receive control input by the user, display feedback to the user, transmit a signal to a remote device, or receive a signal from a remote device. Control input available to the user may include some or all of power on/off, volume, passband characteristics, signal processor in/out, power saving mode, audio compression on/off, audio compression magnitude, signal capture trigger, signal transmit, signal receive, channel select, sample residual analysis on/off, sample residual play, sample residual/library residual toggle, library residual increment, residual play/residual superimpose toggle, and residual/sample balance. Controller 1103 may also by controlled by computer input/ouput using a mouse, voice recognition, etc. Additionally, control circuitry 1002 may include provision for indicator signals including display, LED, and audio.

Signals transmitted between pre-amplifier 1101 and controller 1103, between signal processor 1102 and controller 1103, between amplifier 1001 and controller 1103, and between I/O block 1104 and controller 1103 may comprise, for instance digital control signals such as enable lines and control bytes, analog signals representing audio signals, digital signals representing audio signals, and power lines. Power supply 111 may comprise batteries or other conventional power sources for powering the auscultation aid. Such power may be selectively switched by controller 1103 upon user or automatic input to controller 1103 via I/O block 1104, pre-amplifier 1101, or signal processor 1102.

In some embodiments, such input may comprise manual turn-on by a user activating a power button. Power may be automatically shut off to conserve battery life after a pre-determined period of time. Additionally, logic may switch between power-saving and operation modes. In the latter case, automatic detection of the operational condition of the auscultation aid may determine when to toggle these modes.

In some embodiments, the functions of some of the components shown in FIGS. 10 and 11 may be combined. For example, microphone 109 may be combined with an integrated pre-amplifier 1101. Speaker 112 may be combined with an integrated amplifier 1001. Components such as these are available, for instance, in the hearing aid trade and may be especially useful for minimizing the size of an auscultation aid.

FIG. 12 illustrates the operation of one automatic power-saving approach. I/O block 1104 includes a signal emitter 1201 such as a light-emitting diode (LED) or ultrasonic emitter (for “active sonar”) and a signal detector 1205 corresponding to the signal emitted by signal emitter 1201. In the case where signal emitter 1201 is an infrared (IR) LED, signal detector 1205 could be a photo-diode with sensitivity comprising the IR wavelength emitted by signal emitter 1201. In operation, signal emitter 1201 can emit interrogation signal 1202. In the case where target surface 1203, for instance the surface of an auscultation subject, is present in a position for listening, the surface 1203 reflects interrogation signal 1202 as detection signal 1204. Signal detector 1205 identifies the reflected interrogation signal and provides a corresponding output signal to controller 1103. Controller 1103 detects a ready condition and switches the auscultation aid into an “on” condition by powering-up all necessary circuitry. On the other hand, in the absence of surface 1203, interrogation signal 1202 emits beyond the detection range of detector 1205 as indicated by signal 1206. This situation corresponds to a not ready condition and causes controller 1103 to switch the auscultation aid into a “standby” condition, thus reducing power consumption. Accordingly, the auscultation aid can conserve power through what is effectively a proximity detector. Other types of proximity detectors, such as pressure switches or position sensors may similarly activate the auscultation aid. In addition to saving power, a proximity detector may reduce interference with EKG, ultrasound, or other equipment.

While in standby, the detection apparatus of FIG. 12 may be pulsed intermittently, and thus itself operate in a manner that conserves power. To improve immunity to ambient conditions, emitter 1201 may by cycled relatively rapidly to eliminate “zero drift”.

While in an on condition, the apparatus of FIG. 12 may be operated with a time-constant that keeps the auscultation aid turned on for some time while not in a position to receive sounds form surface 1203. This, for instance, may reduce the incidence of rapid cycling between stand-by and on conditions while changing auscultation locations.

In another power-saving embodiment (“passive sonar”), controller 1103 may sample a signal via microphone 109. FIG. 13 illustrates a characteristic audio signal corresponding to a standby condition 1301 and a characteristic audio signal corresponding to an on condition 1302. Signals 1301 and 1302 illustrate RMS received audio signal versus frequency. Note that when the auscultation aid is positioned in the open air, corresponding to signal 1301, audio power is relatively high and the power is received over a relatively broad frequency range. Conversely, when the microphone is positioned against the auscultation subject and the air column therebetween is sealed, the received audio power is both reduced and high frequency components are reduced as illustrated by curve 1302. The signal level, its spectrum, or both may be used to detect the condition of the auscultation aid and control toggling between stand-by and on conditions. Furthermore, this apparatus and method may be used in conjunction with the apparatus and method illustrated by FIG. 12.

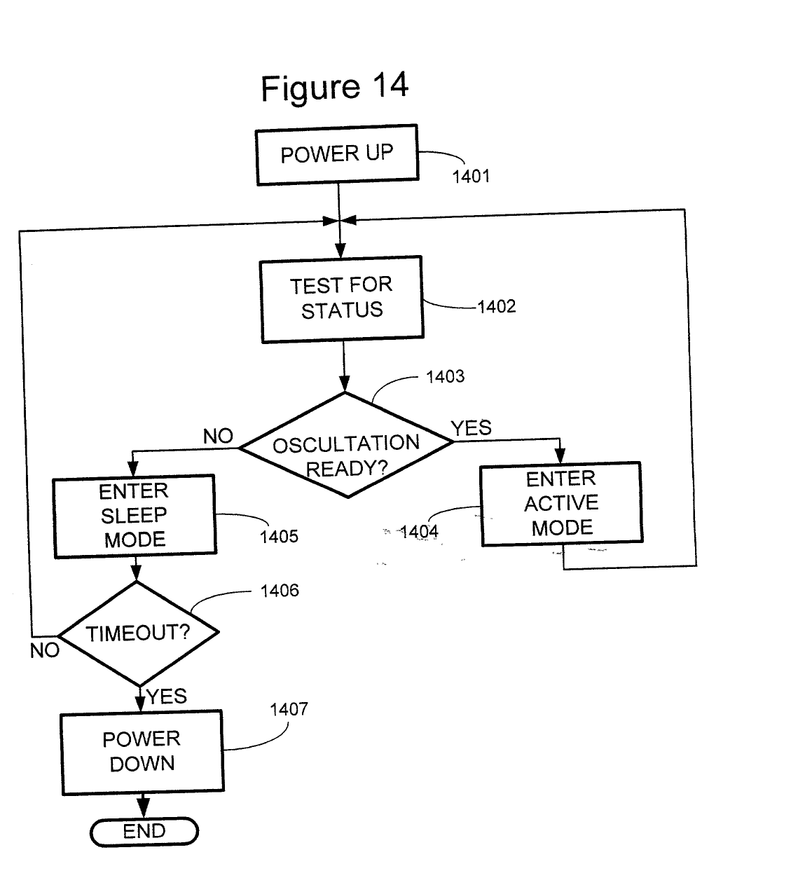

FIG. 14 is a flow chart that illustrates an implementation of power-saving logic for use in conjunction with the aforementioned detection methods and apparatuses. After the auscultation aid is powered-up, for instance in response to the user pushing an “ON” button, by external command via I/O block 1104, or by installing batteries 111, status test 1402 is performed. In the case where an IR emitter-detector pair is used, this test may involve monitoring of signal level received by photo-detector 1205. If emitter light is substantially reflected, then the apparatus is deemed ready for auscultation as shown by decision step 1403, and the controller enters active mode as per block 1404 wherein the auscultation aid is powered to provide operation as selected by the user. In many cases, this means that signal processor 1102 is powered to provide signal processing as selected by the user and power amplifier 1001 is powered to provide output through speaker 112 at a level determined by the user. While in active mode, status test 1402 may be repeated, either constantly or intermittently to determine if the device should continue in the ON condition. Such testing may take place over a number of instances, to allow for brief periods out of auscultation range, before decision block 1403 determines the device is no longer ready for auscultation.

Upon determining that the auscultation aid is not in auscultation position, for instance by determining that a low level of IR signal has be detected by detector 1205, the device may, depending upon user preferences, enter sleep or stand-by mode. In stand-by mode, portions of the circuit may be shut down to reduce power consumption and/or portions may be operated in a mode that reduces power consumption. Intermittently or continuously thereafter, status testing may be repeated to determine if the device has entered a position associated with active mode. Optionally, a timer may be activated to determine an amount of time, number of clock cycles, number of system ticks, number of tests, or the like that has elapsed since entering sleep mode. If optional test 1406 determines that a sufficient number of events have occurred, the auscultation aid may optionally be completely powered down as indicated by process 1407. In the case where a power-down condition is entered, processes shown in FIG. 14 are suspended and an external event indicated by block 1401 may be necessary to restart the process.

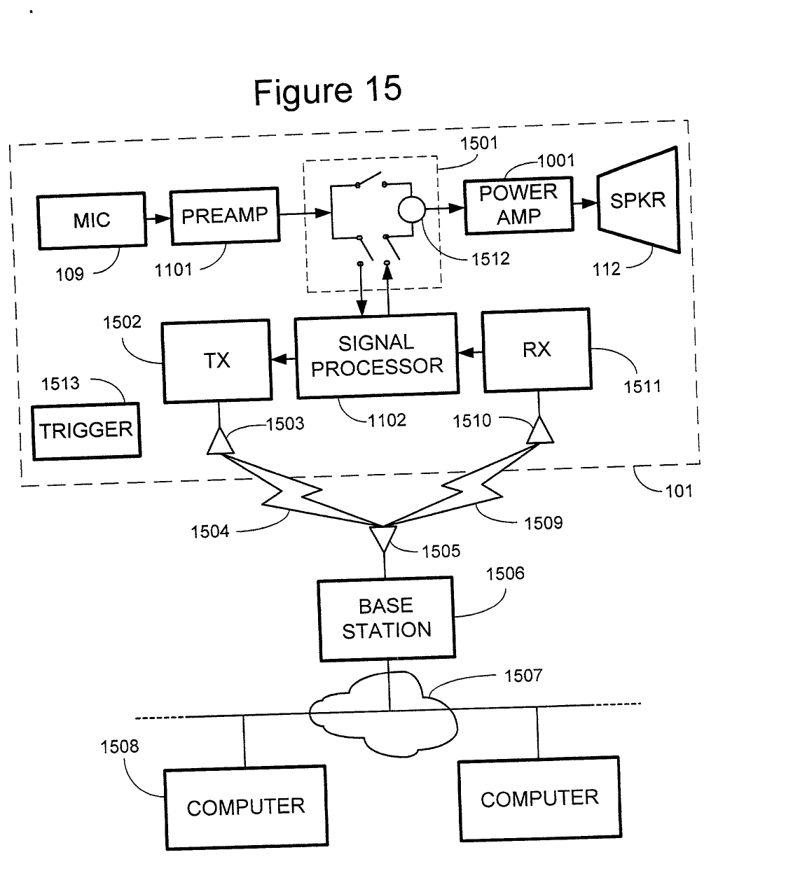

FIG. 15 is a block diagram of an embodiment that allows for remote control, remote signal processing, or input of remotely generated audio signals. Switch block 1501 may be positioned to receive a pre-amplified output signal from pre-amplifier 1101. In one mode, the input signal may be connected directly to power amplifier 1001 for reproduction by speaker 112. In another mode, the input signal may be routed through signal processor 1102 for signal conditioning prior to being sent back to the power amplifier 1001 by switch block 1501. In another mode, the input signal may split, a first portion being sent to mixer 1512, and a second portion being routed through signal processor 1102 prior to being remixed with the input signal in mixer 1512. Mixer 1512 may include a synchronization apparatus to account for propagation and/or processing delay differences between the signal paths. In another mode, all or a portion of output from pre-amplifier 1501 may be routed through a transmitter 1502 and antenna 1503 to a remote antenna 1505 and remote receiver 1506. This may be performed, for instance, by converting the auscultation signal to a streaming format such as MPS. Remote receiver 1506 may optionally be coupled to a computer network 1507 to one or more remote computers 1508.

In one mode, the signal received by remote device 1508 may be used to select operational modes for the auscultation device. In this mode, control signals for selecting operating characteristics of the auscultation aid may be passed to signal processor 1102 via remote link 1509, through receiving antenna 1510 and through receiver 1511.

In another mode, remote device 1508 may perform remote signal processing and pass a processed signal back to local signal processor 1102 for mixing at mixer 1512 via link 1509. One use for this mode is for comparison to and presentation of library images of stored auscultation profiles. This can enable the user of the auscultation aid to compare the detected signal against known pathologies, for instance alternately listening to the subject signal and switching through one or more reference signals. Alternatively, information from the reference signal can be used to boost a portion of the live signal to draw attention to that portion that may be associated with a pathology. Alternatively, the user may elect to play a live residual signal alone as a way of eliminating signals associated with normal body functions.

Trigger 1513 may be used to conveniently enable transmit/receive or to select auscultation profiles or toggle between modes.

Mixer 1512 may be used to synchronize signals passed through the top circuit of switch 1501 and those that pass through signal processor 1102. Mixer 1512 may be further used to select balance between the two signals. In some embodiments, a direct signal from the pre-amplifier may be played through a first speaker 112 into a first binaural and a second signal from signal processor 1102 into a second binaural through a second speaker. This arrangement allows the user to hear a live signal through one ear and a processed or reference signal through the other ear.

Output signal 1504 and input signal 1509 comprise a bidirectional external link. As alternatives to radio links, links 1504 and 1509 may comprise IR signals or may comprise a wired connection.

In some embodiments, an external link 1509 may be used to transmit oral instructions to the user through speaker 112. This may be especially advantageous for cases involving local auscultation efforts by a local practitioner with remote guidance by an expert practitioner.

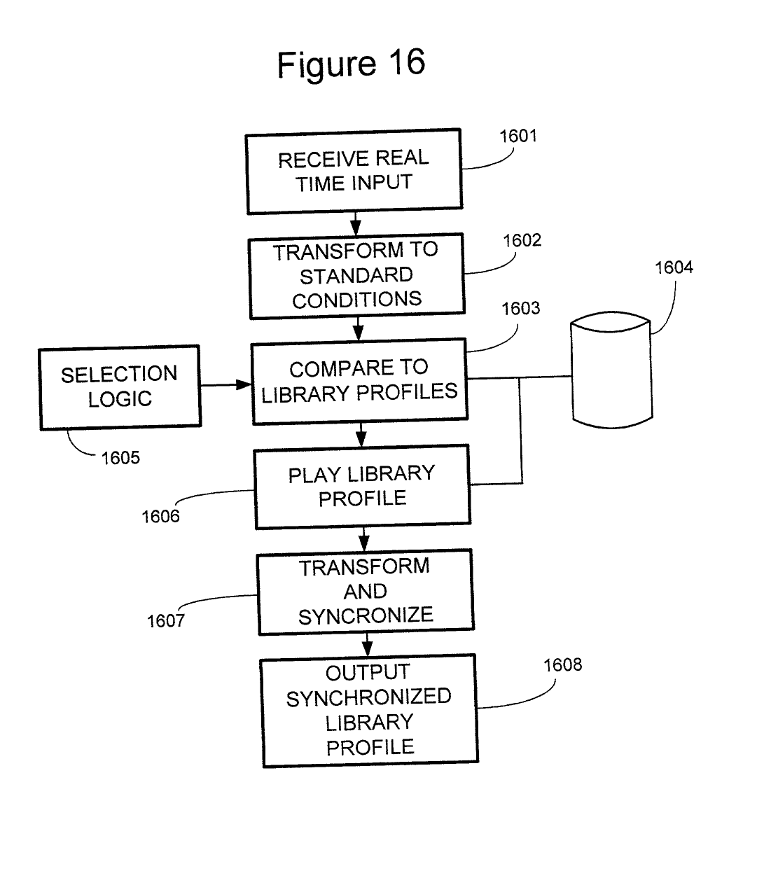

FIG. 16 is a flow chart illustrating a method for comparing a sample auscultation signal against a library of signals characteristic of pathological and pre-pathological conditions for the purpose of aiding diagnosis. An input signal is received via microphone 109 (not shown) in block 1601. The sampling period may be selected to contain at least a full cycle of the sounds of interest. For example, depending upon the nature of the auscultation, a full cycle may comprise a full heartbeat cycle, a full respiration cycle, or some other period. In some applications, the sample auscultation signal may be a portion of the detected signal as split, for instance, using the apparatus of FIG. 15. Next, the input signal is transformed to a set of standard conditions in block 1602. This may include changing the period or frequency of the input signal to match one or more of a set of standard conditions stored in profile library 1604. For instance, if library 1604 contains a full set of conditions at heart rates of 60, 65, 70, etc. beats per minute and the input signal is determined to correspond to 67 beats per minute; then the input signal may be electronically altered to raise its apparent heart rate to 70 beats per minute. This results in matching the input signal to a library standard condition. In general, it is preferable to raise the frequency (reduce the period) of the input signal to allow for later recombination without losing information. The input signal may also differ in relative volume or equalization from library standards. These differences may similarly be compensated for by transforming the input standard using equalization.

After compensation, the input signal is compared, for instance sequentially, against profile signals stored in library 1604. Selection logic 1605 may serve to select most likely matches first and may ultimately select the best fit from an array of choices. A best-fit library profile is played in block 1606, which assembles one or a few periods of the library profile into a substantially continuously repeating pattern. The selected library signal is transformed and synchronized to a best match with the input signal using an inverse of the transformation performed in block 1602. Thus, according to the example described above, the 70 beat-per-minute library signal may be slowed down to match the 67 beat-per-minute input signal with each successive period being synchronized to the input signal. Similarly, any equalization performed in block 1602 may be inverted to produce a transformation of a library signal that is matched to the input signal. The matched output signal may then be played through speaker 112. Such a signal allows the user to compare known pathological or pre-pathological conditions against the auscultation subject. In practice, the user may toggle between the live signal and playback signal for rapid comparison and may increment through a series of best-fit library signals, each optionally dynamically matched to the present conditions of the input signal.

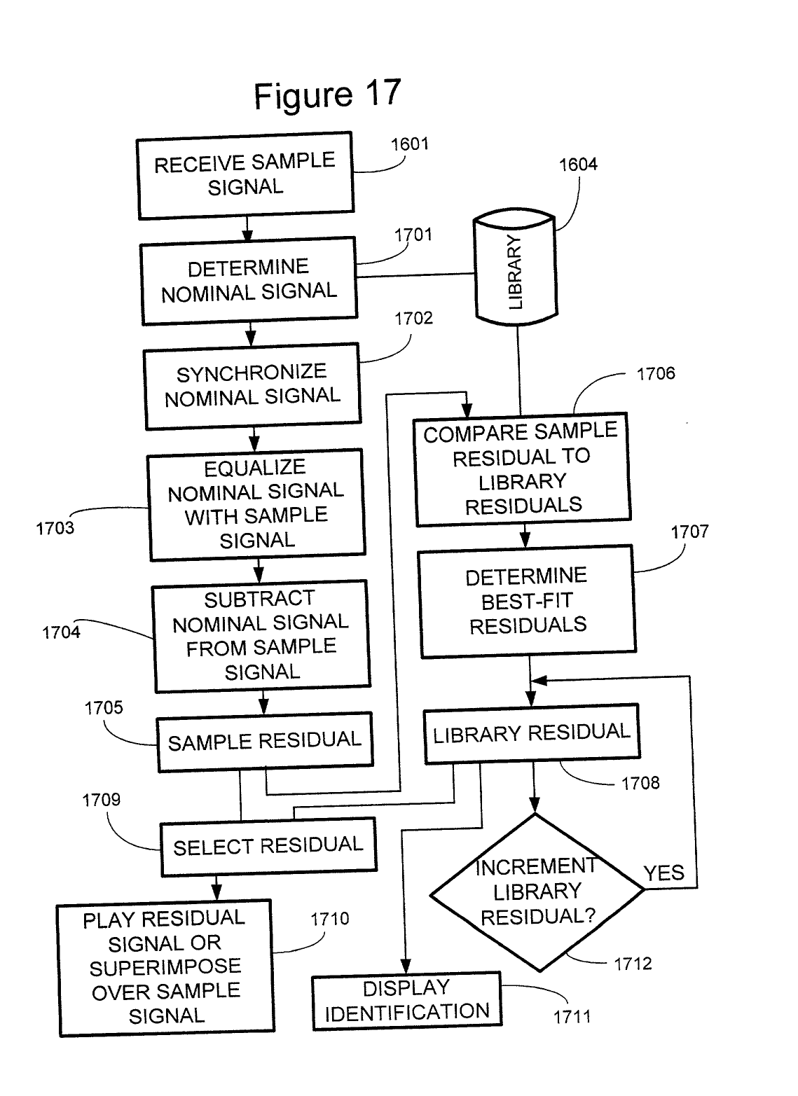

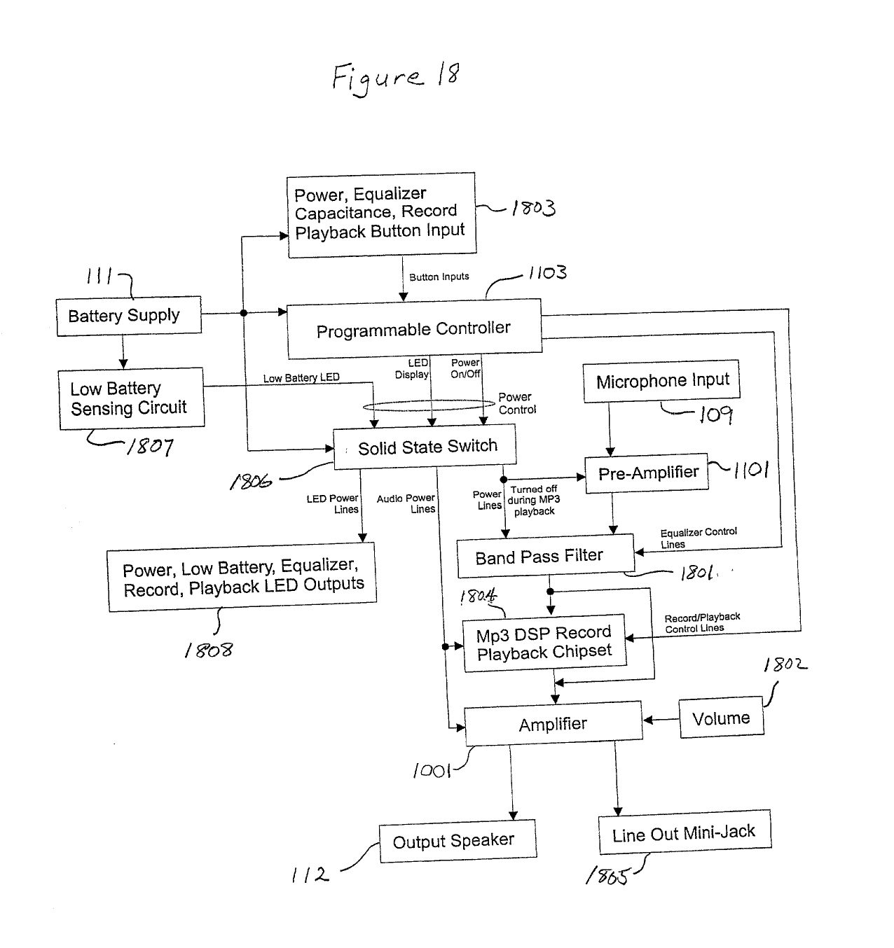

FIG. 17 illustrates a method of transforming a sample auscultation signal to emphasize non-nominal components and optionally for comparing the non-nominal components to a library of pathological and pre-pathological components. In step 1601, a sample signal is received. This may be, for instance, a continuously streaming signal received from pre-amplifier 1101 and optionally converted into a digital format. Next, as shown by step 1701, a nominal signal representing a best-fit baseline pattern is determined. This may be done, for instance, by selecting from a plurality of possible nominal signals stored in library 1604. Such possible matches may be stored in uncompressed or in compressed form in library 1604 so as allow direct selection of a nominal signal. Alternatively or in combination, one or more nominal signals stored in library 1604 may be transformed to match amplitude, equalization, and period of the received sample signal. Alternatively, a nominal signal may be generated algorithmically, optionally as a function of the sample signal itself. A nominal signal represents a healthy signal for an individual matching the characteristics of the auscultation subject, for instance matching the auscultation location, the age, gender, and body weight of the subject.

The nominal signal is synchronized to the input signal in step 1702 to match a repeating nominal signal to the phase of the input signal and optionally to track any changes in input signal period or other characteristics.

Next, the nominal signal may be mathematically combined with the input signal as indicated in step 1704. In one embodiment, the nominal signal may be inverted and added to the input signal to produce a sample residual signal 1705. Sample residual signal 1705 thus represents non-nominal characteristics of the sample signal. These may include, for instance heart murmurs, implanted heart valve sounds, bronchial wheezing, high frequency whistling caused by air movement past an obstruction, or other pathological and pre-pathological conditions. Similarly, the input signal can be signal processed by identifying the spectral content of the non-nominal characteristics and filtering or otherwise reducing the nominal components relative to the non-nominal characteristics.

In the present embodiment, sample residual 1705 is sequentially compared against library residual signals in step 1706. As with the sample residual 1705, library residuals represent differences from nominal. These may include a wide array of known pathological and pre-pathological conditions to which the sample residual is compared. Library residuals that most closely match the sample residual signals are selected using one or more fitting algorithms in step 1707. Such fitting algorithms may, for instance, include least-squares fit, analysis of variance, or other known techniques. A best-fit library residual 1708 is selected by step 1707.

Having a sample residual 1705 and a library residual 1708 available, the user may toggle between the residuals. The selected residual may be superimposed over the sample signal or may be played alone as shown by step 1710. In the case where the sample residual is selected, the superposition of signals has the effect of emphasizing those aspects of the sample signal that are non-nominal or abnormal. For instance, mechanical heart valve clicking could be made somewhat louder than in the original input signal so as to draw attention to such sounds. Similarly, other abnormalities could also be emphasized. For the case where the residual signal is selected to be played alone, the effect is one of active noise cancellation wherein the sounds of normal body functions are attenuated relative to the sounds associated with a selected pathology or pre-pathology. In this mode, the act of comparing the sample residuals to library residuals is optional. Simply the act of attenuating normal sounds with a stored or synthesized nominal signal has the effect of drawing the practitioner's attention to unusual auscultation features.

For the case where the library residual is selected, non-nominal or abnormal sounds characteristics of a known pathological or pre-pathological condition may be superimposed over the input signal. By toggling back and forth between the sample residual and library residual, the user may rapidly and conveniently compare the auscultation subject's heart, lung, or other sounds to those of a known pathology. The known pathology may be displayed to the user, shown in step 1711, using a local or remote display, for instance an LCD.

The user may also increment and decrement between best-fit library residuals as shown in step 1712. Thus, the user may compare the auscultation subject's sounds against a plurality of most likely pathological and pre-pathological conditions and, in effect, receive expert consultation even for obscure conditions that might otherwise be missed.

FIG. 18 is a block diagram of an embodiment of the present invention having record and playback functionality. As with earlier block diagrams, low-level signals from microphone 109 are amplified to usable levels by pre-amplifier 1101. Pre-amplifier 1101 outputs to band pass filter 1801. Band pass filter 1801 may optionally be programmable via equalizer control lines from programmable controller 1103. During normal operation, output from band pass filter 1801 is sent to power amplifier 1001 and on to output speaker 112 which may be placed to as to emit into one or more air columns of a stethoscope. Optionally, line-out jack 1805 may be used to connect the output signal to one or more external devices, either in place of outputting through output speaker 112 or in parallel to output speaker 112. Volume control 1802 may be used to control the gain of power amplifier 1001, and hence adjust for a comfortable listening level.

Programmable controller 1103, upon activation by record or playback controls 1803, may optionally enable record and playback apparatus 1804 to capture an auscultation signal from band pass filter 1801. In some embodiments, record and playback apparatus 1804 may provisionally store an auscultation signal on, for instance, a first-in, first-out basis. Upon receipt of a record trigger from the user or upon signal matching to a library profile, a non-volatile recording may then be made including a portion of signal from before receipt of the trigger. This feature can be useful for capturing signals that include events leading up to triggering events as well as post-trigger signals. Upon receipt of a record command or trigger, record and playback apparatus may capture a period of auscultation signal. Subsequently, a playback command may cause the recorded signal to be played back through output speaker 112 and/or through line out jack 1805 for distribution to one or more external devices. Playback through line out jack 1805 may be useful, for instance, for consultation purposes, for teaching purposes, and for medical record-keeping purposes. In some embodiments record and play apparatus 1804 may record images of auscultation signals in a streaming data format or a standard streaming data format. In some embodiments, record and play apparatus 1804 may record in an MP3 digital audio format. Optionally, programmable controller 1103 may transmit a digital recording such as an MP3 recording directly to one or more external devices.

During playback, pre-amplifier 1101 and band pass filter 1801 may be shut off to avoid injection of a spurious auscultation signal and for power saving. Powering of various components of the embodiment of FIG. 18 may be controlled via switch 1806 under command from programmable controller 1103 and optionally directly from power button 1803 and optionally by low battery sensing circuit 1807. When low battery sensing circuit senses low voltage in battery 111, it may optionally shut off power via solid state switch 1806 to avoid placing components in ambiguous logic states. Subsequent connection to external power, replacement of batteries, or recharging of batteries may optionally cause low battery sensing circuit 1807 to re-enable switch 1806 to provide power to selected components.

Indicator devices 1808 may be used to display device operation mode and device status to the user. In some embodiments, indicator devices 1808 may comprise an array of LEDs. In other embodiments, indicator devices 1808 may comprise an LCD, a plasma display, a field-emissive display, a cathode ray tube, a retinal scanning display, or other known display device.

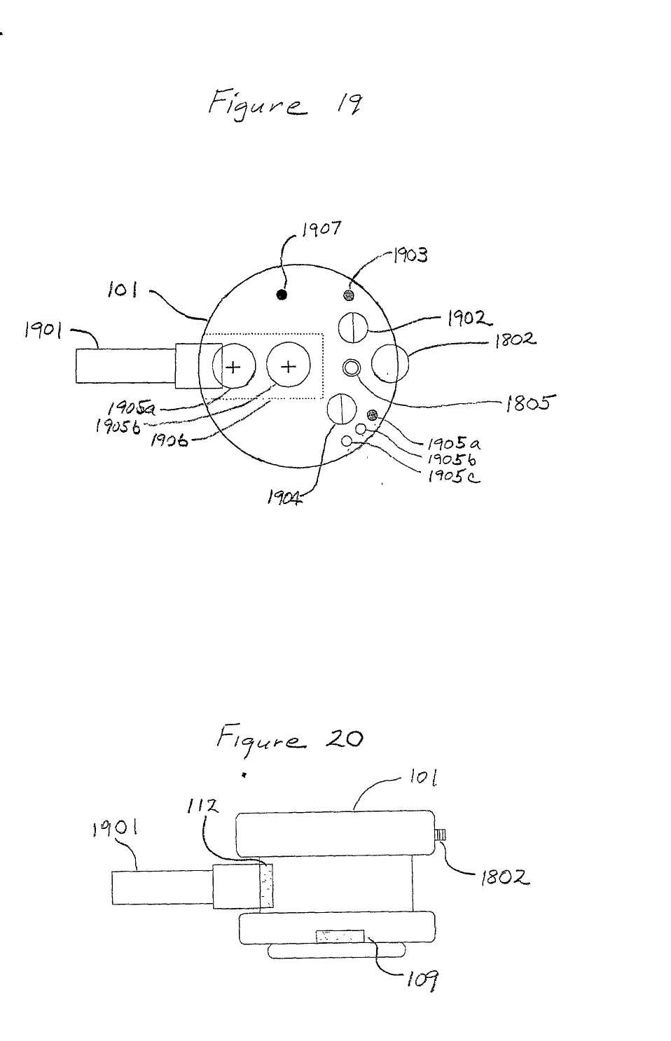

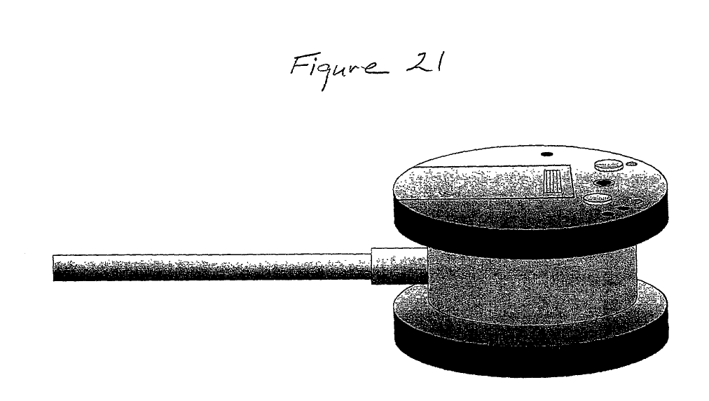

FIGS. 19, 20, and 21 present various user controls for an embodiment of the present invention. Monaural 1901 is attached to housing 101 in a position to receive output from output speaker 112. Power button 1902 switches on the electronics, for instance electronics corresponding to FIG. 18. Power indicator 1903 may be an LED that is illuminated when power is turned on. Equalizer select switch 1904 may be a momentary switch that toggles between equalizer modes. Equalizer lamps 1905a, 1905b, and 1905c, which may be LEDs, indicate the equalizer mode to the user. Equalizer indicator lamps 1905a, 1905b, and 1905c may be illuminated singly or in combination to indicate equalizer modes. Volume control 1802 is a rotary control in this embodiment that may be turned clockwise to increase output volume or counterclockwise to decrease volume. In some embodiments, rotary control 1802 may be connected to a rotary encoder. Line out jack 1805 may be an RCA-type mini-jack for connecting to any device capable of receiving signals through an RCA cable. In operation, line out jack 1805 may be connected to, for instance, head phones, ear phones, a microphone input of a recording device, a microphone input to a computer, laptop computer, or wearable computer, external speaker, external wireless transmitter, or other device. Batteries 1905a and 1905b are shown with battery cover 1906 removed. Batteries 1905a and 1905b may, for instance be coin cells. Low battery indicator lamp 1907, which may be, for instance an LED, illuminates when battery voltage sags to a pre-determined warning level to advise the user of the need to recharge or replace batteries 1905a and 1905b.

FIG. 20 is a side depiction of the embodiment diagrammatically indicated by FIG. 19. Additionally, FIG. 20 shows hidden features including microphone 109 and output speaker 112. The apparatus of FIGS. 19 and 20 represents an alternative embodiment of a detachable auscultation aid wherein the housing 101 may be removably attached directly to a monaural tube 1901 of an acoustic stethoscope in lieu of an acoustic bell assembly. Alternatively, housing 101 may be attached directly to binaural tubes of a binaural acoustic stethoscope. Alternatively, housing 101 may be permanently attached to monaural or binaural tubes, making the device of FIGS. 19 and 20 an electronic stethoscope with no acoustic bell. In this case, the entire electronic stethoscope may be termed an auscultation aid.

FIG. 22 is a diagram of an electronic auscultation aid that may be inserted into the air column of an acoustic stethoscope. Housing 2201 has a first end 2203 with fitting 2204 and second end 2205 with fitting 2206, each fitting having a provision for mounting the housing into the air column of an acoustic stethoscope. A microphone 109 is mounted to receive sounds through fitting 2204. A speaker 112 is mounted to emit sounds through fitting 2206. In one embodiment, the microphone 109 may include an integrated pre-amplifier 1101 (not shown) and speaker 112 may include an integrated amplifier 1001 (not shown). Disposed within housing 2201 are batteries and electronics for transmitting and conditioning a signal from microphone 109 to speaker 112 as shown in other figures. User controls 1802 and 1904, for instance, and indicators 1905a, 1905b, and 1905c, for instance, may be mounted on the side of housing 2201.

In some embodiments, fittings 2204 and 2206 comprise barbed couplings for inserting into the ends of tubes 2207 and 2208, respectively. In other embodiments, fittings 2204 and 2206 comprise detachable fittings whereby the electronics housing may be conveniently removed, and the ends of the tubes reattached, thus restoring the stethoscope to an acoustic mode. Although FIG. 22 depicts detaching features as screw fittings, other fittings such as Luhr fittings, bayonet fittings, standard air fittings, may be substituted and remain within the spirit and scope of the disclosure.

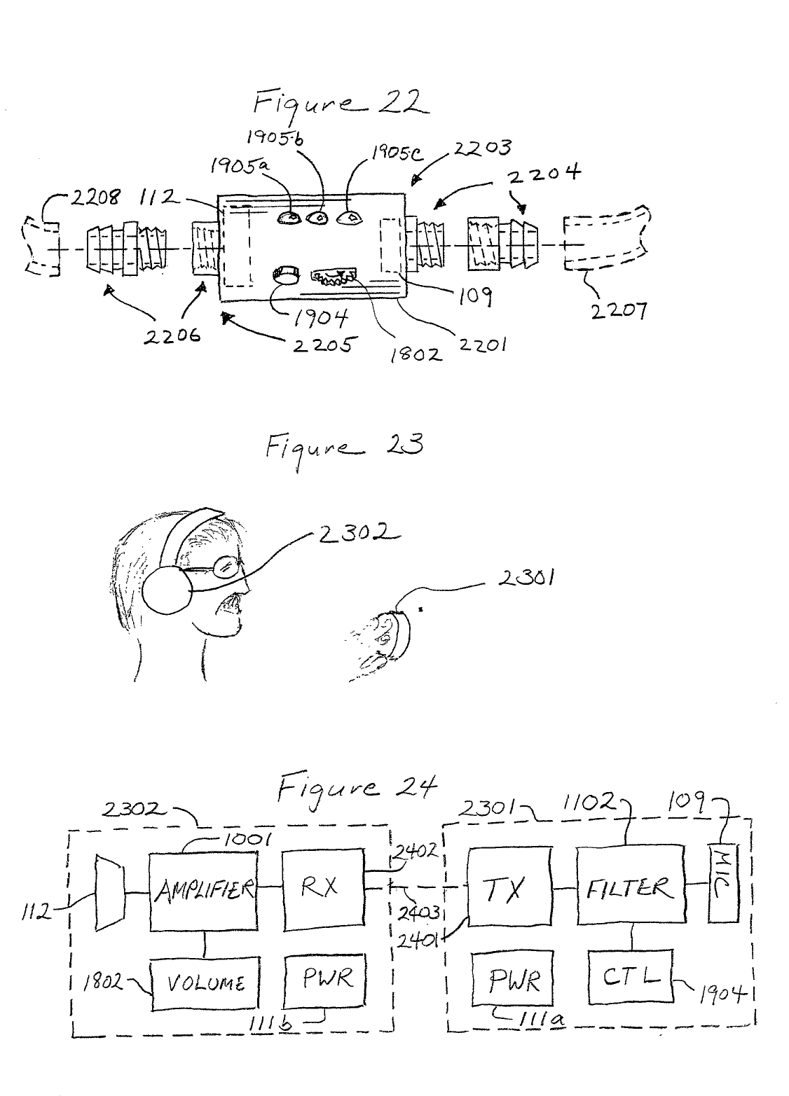

FIG. 23 is a perspective view of an electronic auscultation system comprising a detector portion and a headphone portion. A detector 2301 may be placed to detect sounds. An earpiece 2302 may be worn by the user to listen to sounds detected by detector 2301. Signals representative of sounds are transmitted from detector 2301 to earpiece 2302.

FIG. 24 is a block diagram of an electronic auscultation system having a detector portion and an electronically coupled earpiece portion. Detector2301 comprises a microphone with integrated pre-amplifier 109 connected to optional filter 1102. Optional filter 1102 comprises an equalizer circuit with 2 or more equalization profiles selected by control input 1904. Output from integrated microphone/pre-amplifier 109 or, if present, filter 1102 is sent to transmission component 2401 for transmission to earpiece 2302.

Transmission component 2401 communicates with transmission component 2402 in earpiece 2302 via transmission channel 2403. Several forms of signal transmission are possible. In some embodiments, transmission components 2401 and 2402 comprise simple connectors for transmitting an analog signal carried by a wire. In some embodiments, transmission component 2401 includes an analog-to-digital (A/D) converter and a protocol controller for transmission via a digital signal 2403. In this case, receiver 2402 includes a protocol controller for receiving digital signal 2403 and a digital-to-analog (D/A) converter for converting the signal back to analog. The digital transmission signal may be carried on a wire by, for instance, RS 232 serial, USB, IEEE 1204 parallel, or firewire compatible physical interfaces. Alternatively, digital signal 2403 may comprise a wireless signal using a radio or infrared interface for instance. If using radio, it may be advantageous to use a band allocated for unlicensed use such as 13.5 MHz, 900 MHz, 2.4 GHz, or 5.8 GHz. Also if using a radio interface, it may be desirable to use a standard protocol such as IEEE 802.11, HomeRF, Bluetooth, or other protocol for which standard components are available. If using infrared data transmission, it may be advantageous to use a standard protocol such as IRdA. It is also possible, and may be advantageous for some applications, for a wireless signal 2403 to comprise an analog signal.

Earpiece 2302 may be in the form of a headphone, an earphone, a helmet or other apparatus as preferred according to the application. Alternatively, earpiece 2302 may comprise an open-air speaker, an embodiment that may be particularly useful when it is desirable for multiple listeners to hear detected tones, such as in an auscultation class.

Receiver 2402 receives transmitted signal 2403 and performs any necessary conversion necessary for output. The signal from receiver 2402 is passed to and amplified by amplifier 1001, and subsequently output by speaker 112. In one preferred embodiment, amplifier 1001 and speaker 112 are combined as an integrated assembly. Volume control 1802 is coupled to the signal to produce variable amplification or variable attenuation as preferred by the user for listening comfort. Alternatively, volume control 1802 may be placed on detector portion 2301.

If the embodiment of FIG. 24 uses a wireless interface 2403, detector 2301 and earpiece 2302 may each contain a power source 111a and 111b, respectively. Power sources 111a and 111b may comprise electrochemical cells or batteries.

FIG. 24 includes an earpiece block diagram consistent with a wireless interface. It should be understood that if detector 2301 and 2302 are connected via a wired interface, many of the components illustrated as residing in the earpiece may instead reside in the detector. In particular, if interface 2403 is a wired analog interface, earpiece 2302 may essentially consist of one or more speakers packaged to transmit sound to a user's ears.

In the case where interface 2403 is a wired analog interface, it may be desirable to move control functions including volume control 1802 to the detector portion. While it may be desirable to use an integrated amplifier/speaker, it is also possible to separate their functionalities and place the amplifier 1001 in detector 2301. In this case, transmission medium 2403 would connect amplifier 1001 to speaker 112, in lieu of transmitting the signal between filter 1102 and amplifier 1001. In the case where interface 2403 is a wired interface, power sources may be combined as a single power source 111 residing, for instance, in detector 2301.

The preceding overview of the invention, brief description of the drawings, and detailed description describe exemplary embodiments of the present invention in a manner intended to foster ease of understanding by the reader. Other structures, methods, and equivalents may be within the scope of the invention. As such, the scope of the invention described herein shall be limited only by the claims.

What is claimed is:

1. An auscultation aid, comprising;
a housing,
an audio detector mounted to said housing,
an amplifier within said housing and in communication with said audio detector,
an audio emitter electrically coupled to said amplifier and mounted to said housing, and
a mount on said housing for coupling said housing to an acoustic stethoscope.

2. The auscultation aid of claim 1, wherein;
said mount is adaptable to a plurality of stethoscope shapes and sizes.

3. The auscultation aid of claim 2, wherein said adaptable mount further comprises;
a receiving bore, and
a compressible member mounted therein;
wherein said compressible member may receive a first stethoscope bell.

4. The auscultation aid of claim 3, wherein;
said first compressible member may be interchanged with a second compressible member,
said second compressible member has a dimension different than said first compressible member, and
said second compressible member may receive a second stethoscope bell having a dimension different than said first stethoscope bell.

5. The auscultation aid of claim 3, wherein;
said first compressible member has adhesive thereon for adhering said first compressible member to said receiving bore.

6. The auscultation aid of claim 2, wherein;
said adaptable mount comprises at least one lever,
wherein said lever is positioned to urge a stethoscope bell against said housing.

7. The auscultation aid of claim 6, wherein;
said lever is rotatably coupled to said housing.

8. The auscultation aid of claim 6, wherein;
said lever is elastically coupled to said housing.

9. The auscultation aid of claim 2, wherein;
said adaptable mount comprises a strap for removably coupling said housing to a stethoscope bell.

10. The auscultation aid of claim 2, wherein;
said adaptable mount comprises one or more set-screws for temporarily mounting said housing on the acoustic stethoscope.

11. The auscultation aid of claim 2, wherein;
said adaptable mount comprises an elastic cup for receiving a stethoscope bell.

12. The auscultation aid of claim 1, further comprising;
a bracket for attaching to a bell of a stethoscope,
said bracket comprising;
one or more features for forming a removable coupling to said mount.

13. The auscultation aid of claim 12, wherein;
said removable coupling comprises a screw mount.

14. The auscultation aid of claim 12, wherein;
said removable coupling comprises a bayonet mount.

15. The auscultation aid of claim 12, wherein;
said removable coupling comprises a magnetic mount.

16. The auscultation aid of claim 1, wherein;
said audio detector comprises a microphone.

17. The auscultation aid of claim 1, wherein;
said audio emitter comprises a speaker.

18. A method of aiding an act of auscultation, comprising the steps of;
establishing a sample signal,
determining a nominal signal, and
combining said sample signal and said nominal signal to produce a sample residual signal,
whereby said sample residual signal may be used to emphasize a condition of an auscultation subject.

19. The method of claim 18, wherein;
said step of establishing a sample signal is accomplished by detecting audible tones through a microphone.

20. The method of claim 18, wherein;
said step of establishing a sample signal is accomplished by playing back a recording of audible tones.

21. The method of claim 18, wherein;
said step of determining of a nominal signal is performed by retrieving a nominal signal from a computer memory.

22. The method of claim 21, wherein;
said nominal signal is selected from a plurality of nominal signals stored in computer memory.

23. The method of claim 18, wherein;
said nominal signal is generated by an algorithm that uses characteristics of said sample signal.

24. The method of claim 18, wherein;
said step of combining the sample and nominal signals further comprises the steps of;
inverting said nominal signal to produce an inverted nominal signal, and
superimposing said sample signal and said inverted nominal signal.

25. The method of claim 18, further comprising the steps of;
amplifying said sample residual signal, and
playing said sample residual signal through an output speaker.

26. The method of claim 18, further comprising the steps of;
comparing said sample residual signal to a plurality of library residual signals held in computer memory, and
determining one or more library residual signals that is similar to said sample residual signal, wherein;
each of the one or more library residual signals is characteristic of a condition.

27. The method of claim 26, further comprising the steps of;
selecting between said sample residual signal and said one or more library residual signals, and
playing said sample residual signal or library residual signal through an output speaker.

28. The method of claim 26, further comprising the step of;
making visual indication to the user the identity of the one or more characteristic conditions of which said one or more library residual signals is characteristic.

29. An auscultation aid, comprising;
a microphone for detecting sounds emitted from an auscultation subject,
an amplifier for amplifying said detected sounds as an electrical signal, and
a wireless interface for transmitting said electrical signal to a remote device.

30. The auscultation aid of claim 29, wherein;
said wireless interface comprises a radio interface.

31. A storage device for storing patient auscultation records, comprising;
a receiver for receiving an electrical signal representative of sounds collected from an auscultation subject,
a storage medium for recording said electrical signal.

32. The storage device of claim 31, wherein;
said receiver is a radio receiver.

33. An auscultation device, comprising;
a microphone,
an amplifier for amplifying signals from said microphone,
an emitter-detector pair for determining when said microphone is in a position to detect sounds from an auscultation subject, and
a controller for powering down said amplifier when said emitter-detector pair indicate the auscultation device is not ready to detect sounds from said auscultation subject.

34. An auscultation device, comprising;
a housing having a first and a second end,
a microphone carried within said housing and arranged to detect audio signals arriving at the first end of said housing,
an amplifier carried within said housing and electrically coupled to said microphone,
a speaker carried within said housing arranged to emit audio signals at the second end of said housing and coupled to the output of said amplifier,
whereby;
said housing may be coupled into an air column of a stethoscope.

35. The auscultation device of claim 34, further comprising;
a fitting on the first end of said housing for coupling to a first stethoscope tube connected to a bell, and
a fitting on the second end of said housing for coupling to a second stethoscope tube coupled to an earpiece.

36. The auscultation device of claim 35, further comprising;
a bell,
a first tube coupling said bell to the first end of said housing,
a second tube having a first and second end, the first end being coupled to the second end of said housing, and
an earpiece coupled to the second end of said second tube.

37. The auscultation device of claim 35, wherein;
said fittings comprise barbed fittings.

38. The auscultation device of claim 35, wherein;
said fittings comprise a mated pair, whereby;
the first tube and second tube may be coupled together when said housing is removed, thus reverting the stethoscope to an acoustic stethoscope.

39. The auscultation device of claim 35, wherein;
said fittings comprise bayonet mount fittings.

40. The auscultation device of claim 35, wherein;
said fittings comprise screw fittings.

41. The auscultation device of claim 34, further comprising;
a signal processor coupled between said amplifier and said speaker for modifying the sonic characteristics of the audio signal output by the speaker.

42. The auscultation device of claim 34, further comprising;
user controls mounted on the side of said housing for controlling the operational characteristics of the electronics housed therein.

43. An auscultation device, comprising;
a detector for detecting sounds,
an interface for transmitting an electrical representation of detected sounds from said detector, and
an earpiece for receiving the electrical representation of detected sounds from said interface and reproducing an audio signal therefrom.

44. The auscultation device of claim 43, wherein;
said interface comprises two or more wires.

45. The auscultation device of claim 43, wherein;
said interface comprises a radio interface.

46. The auscultation device of claim 43, wherein;
said interface comprises an infrared interface.
